(19) United States
(12) Patent Application Publication
(10) Pub. No.: US 2025/0114644 A1
(43) Pub. Date: Apr. 10, 2025

(54) LONG-TERM PRECISE ULTRASOUND NEUROMODULATION VIA STABLE HOLLOW SILICA NANOPARTICLES

(71) Applicant: THE HONG KONG POLYTECHNIC UNIVERSITY, Hong Kong (CN)

(72) Inventors: Lei SUN, Hong Kong (CN); Xuandi HOU, Hong Kong (CN); Jianing JING, Hong Kong (CN); Yizhou JIANG, Hong Kong (CN)

(21) Appl. No.: 18/483,109

(22) Filed: Oct. 9, 2023

(51) Int. Cl. A61N 7/00

(52) U.S. Cl. CPC A61N 7/00; A61N 2007/0026

(57) ABSTRACT

The present invention provides an ultrasound neuromodulation actuator including a stable hollow silica nanoparticle (HSN) structure, in particular, a PEGylated HSN (pHSN) particle, and a method for preparing the same. The present invention also provides methods for enhancing ultrasound stimulation and treating neurological disorders of a subject including applying the pHSN particles to a brain region of the subject followed by ultrasound stimulation.

### TECHNICAL FIELD

The present invention relates to a material as a long-term precise ultrasound neuromodulation actuator, in particular, modified hollow silica nanoparticles with enhanced biocompatibility and stability for long-term precise ultrasound neuromodulation.

### BACKGROUND

Ultrasound is a form of mechanical energy that has been routinely used alone or in combination with certain ultrasound contrast agents such as microbubbles and nanobubbles for diagnostic imaging. Since ultrasound wave is capable to pass through skull of various animals to reach deep regions of their brain, it spurs many researchers to explore its potentials in neuromodulation and treating neurological disorders. Various brain regions of many animal species such as rodents, rabbits, pigs, sheep and non-human primates have been shown to be successfully stimulated by low-intensity ultrasound. Those brain regions include, but not limited to, thalamus, prefrontal, visual, motor and somatosensory cortices. Some studies also suggested it is a possible treatment for a wide range of neurological disorders such as Alzheimer's disease, Parkinson's disease, epilepsy, depression, and amyotrophic lateral sclerosis. It has been shown in some studies that ultrasound can affect the functioning of the central nervous system without significant accompanying damage. In this regard, although one major attribute of ultrasound is to provide good spatial resolutions for deep regions of the brain of the test subject without surgical invasion, a corresponding diffraction-limited spatial resolution must be compromised within millimeters to centimeters range in order to enable low-frequency ultrasound to pass through an intact skull of the test subject.

To solve the aforementioned problem, some studies employed microbubbles, nanobubbles or soft-shelled structures as localized actuators to enhance ultrasound effects on modulation of different cellular activities. However, these conventional bubbles (micro- and nano-bubbles) and soft-shelled structures still suffer from rapid clearance from corresponding site(s) of injection, causing a short lifetime in vivo, thereby limiting their applications.

Some recent studies have successfully demonstrated that hollow silica nanoparticles (HSN) can produce ultrasound contrast and exhibit harmonic scattering to enable enhanced detection versus background in vivo. However, similar to other silica materials, most HSN cannot disperse well in solution, hindering their potentials in practical applications.

A need therefore exists for an improved material with enhanced stability and biocompatibility after injection to a target site for long-term precise ultrasound neuromodulation that at least diminishes or eliminates the disadvantages and problems described above.

### SUMMARY OF INVENTION

Accordingly, a stable and biocompatible material that can disperse well in a medium or suspension for cell culture or in vivo administration, oscillate under ultrasound field, and induce stable cavitation is provided. The present material should be capable to facilitate, assist, or synergistically enhance ultrasound stimulation applied to a subject in order to control certain cellular signalling mechanisms and also chronic stimulation to certain targeted brain regions of the subject. The present material should also be capable to stay in the site of administration or targeted tissue/regions of ultrasound stimulation for a reasonably long period of time without causing any damage to corresponding tissues/regions and toxicity effect on the subject.

Therefore, in a first aspect, the present invention provides an ultrasound neuromodulation actuator comprising a modified hollow silica nanomaterial. Exemplarily, the modified hollow silica nanomaterial is a surface-modified with Polyethylene glycol (PEG) to form PEGylated hollow silica nanomaterial (pHSN).

In certain embodiments, the surface-modified HSN structure is in particle form with an average diameter from 200 to 300 nm.

In certain embodiments, the surface-modified HSN shows good ultrasound contrast signals.

The present invention provides a method for preparing the modified hollow silica nanomaterial and the method includes at least the following steps:

mixing the silica nanoparticle precursor, structure enhancer, and polymer beads to form a plurality of core-shell particles;

collecting and washing the plurality of core-shell particles;

calcinating the plurality of core-shell particles to obtain a plurality of hollow silica nanoparticles (HSN);

modifying a surface of each of the plurality of HSN by one or more reactive groups in order to obtain a plurality of surface-modified HSN.

In certain embodiments, the hollow silica nanoparticle precursor is a mixture of tetramethyl orthosilicate (TMOS) and trimethyloxyphenylsilane (TMPS) and the solvent is ethanol.

In certain embodiments, the hollow silica nanoparticle structure enhancer is iron (III) ethoxide.

In certain embodiments, the plurality of silica particles is collected by centrifugation after said reacting the plurality of cationic polymer beads with the second solution followed by washing with ethanol.

In certain embodiments, said calcinating the plurality of core-shell particles is performed in an atmospheric air within a muffle furnace under an elevating temperature starting from room temperature to approximately 550° C. at a rate of 1.5° C. per minute.

In certain embodiments, said modifying the surface of each of the plurality of HSN particles includes reacting the plurality of core-shell particles after said calcinating with an aminopropyltriethoxysilane-containing solution to obtain a plurality of amino-modified HSN particles, followed by reacting the plurality of amino-modified HSN particles with amine-PEG in a molar ratio and in the presence of a coupling agent and an amine-reactive crosslinking agent.

In certain embodiments, the coupling agent is 1-(3-dimethylaminopropyl)-3-ethylcarbodiimide hydrochloride (EDC) and the amine-reactive crosslinking agent is N-hydroxysuccinimide (NHS).

In certain embodiments, the molar ratio between the plurality of amino-modified HSN particles and the amine-PEG is 1:1.

The PEG surface-modified HSN show better suspension than HSN, indicating the successful modification.

A second aspect of the present invention provides a method for enhancing ultrasound stimulation in neuronal cells, where the method includes using the modified HSN described in the first aspect.

A third aspect of the present invention provides a method for pHSN-actuated localized ultrasound neuromodulation chronically in vivo comprising using the modified HSN described in the first aspect.

In certain embodiments, the modified hollow silica nanoparticles are applied to a brain region of the subject via intra-ventral tegmental area (VTA) administration.

In certain embodiments, the intra-ventral tegmental area (VTA) administration of the modified hollow silica nanoparticles is performed by using a stereotaxic apparatus and the subject is under anesthesia during the intra-VTA administration.

In certain embodiments, ultrasound stimulation to a target site or region of the subject is applied following the intra-VTA administration of the modified hollow silica nanoparticles.

In certain embodiments, the ultrasound stimulation is a low-intensity, low-frequency ultrasound stimulation with an acoustic intensity of smaller than 0.5 MPa.

A fourth aspect of the present invention provides a method for treating neurological disorders, where the method includes administering the modified hollow silica nanoparticles described herein to a subject in need thereof. Alternatively, the modified hollow silica nanoparticles described herein are used in preparation of an injectable formulation for treating neurological disorders in a subject in conjunction with an application of ultrasound stimulation to one or more target regions or sites of administration in the subject.

In certain embodiments, the modified hollow silica nanoparticles or the injectable formulation is administered to the subject via intra-VTA administration.

In certain embodiments, the one or more target regions or sites of administration include striatum in brain regions for ultrasound imaging.

In certain embodiments, the neurological disorders include Alzheimer's disease, Parkinson's disease, epilepsy, depression, and amyotrophic lateral sclerosis.

In certain embodiments, the ultrasound stimulation is a low-intensity, low-frequency ultrasound stimulation with an acoustic intensity of smaller than 0.5 MPa.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows a series of images with a flowchart depicting a method for preparing the PEGylated hollow silica nanoparticles (pHSN) according to certain embodiments;

FIG. 2A shows a transmission electron microscopy (TEM) image of the HSN material prepared according to the method depicted in FIG. 1; scale bar: 200 nm;

FIG. 2B shows a TEM image of the surface-modified HSN material prepared according to the method depicted in FIG. 1; scale bar: 50 nm;

FIG. 3 shows an ultrasound image of the pHSN material prepared according to the method depicted in FIG. 1; scale bar: 1 cm;

FIG. 4 shows a dispersibility of HSN and pHSN particles in a solution at 0, 5, 10, 15, 20, 30, 45, and 60 minutes

FIG. 5 shows an image of customized setup of a passive cavitation detection system for testing the pHSN prepared according to the method depicted in FIG. 1;

FIG. 6 shows an average frequency spectrum of backscattered signals from a suspension of the pHSN measured by the setup depicted in FIG. 5;

FIG. 7 shows a cell viability test result of the pHSN prepared according to the method depicted in FIG. 1 compared with a control sample (without pHSN); bars represent the mean±SEM of four independent experiments; no significant differences (n.s.) were found by t-test;

FIG. 8 schematically depicts a proposed pHSN-mediated ultrasound (US) neuromodulation mechanism according to certain embodiments;

FIG. 9 shows fluorescence images of primary neurons responsive to ultrasound stimulation (US ON) in terms of calcium influx compared to initial baseline when there was no ultrasound stimulation (US OFF) in the presence (+) and absence (−) of the prepared pHSN according to certain embodiments; scale bar: 50 μm;

FIG. 10 shows a time course of the calcium imaging of the primary neurons as shown in FIG. 9 under the ultrasound stimulation in the presence (+) and absence (−) of the prepared pHSN according to certain embodiments; ΔF/F₀: the change in fluorescence/initial baseline;

FIG. 11 shows synchronized neuronal Ca²⁺ responses in terms of the change in fluorescence/initial baseline (ΔF/F₀) under ultrasound stimulation (US) in the presence of pHSN and with/without mechanosensitive ion channel blocker gadolinium (Gd³⁺);

FIG. 12 shows quantification of neuronal Ca²⁺ responses with Gd³⁺ treatment as shown in FIG. 11 compared to those without (CTRL) Gd³⁺ treatment; values are represented by mean s.d. with a p-value of 0.0052;

FIG. 13 schematically depicts a scheme of in vivo ultrasound stimulation (US) in the presence of the prepared pHSN according to certain embodiments, where an inset shows a schematic diagram of how the ultrasound and the prepared pHSN interact with each other at the cellular/neuronal level in an animal model at the target site of stimulation/application of the prepared pHSN; the blue circle indicates the site of administration of the prepared pHSN;

FIG. 14 shows merged fluorescence images of brain sections from the animal model depicted in FIG. 13 under ultrasound stimulation (US) injected with the prepared pHSN (+pHSN) or without the pHSN (saline only); DAPI stains (blue) represent living neurons/cells; c-FOS expression (red) represent activated neurons; yellow dotted square defines region or site of injection of the prepared pHSN or saline, which corresponds to the ventral tegmental area (VTA) of the brain from the animal model;

FIG. 15 shows site of injection (left panel) and ultrasound images of mouse brain injected with pHSN on Day 1 (middle panel) and on Day 21 (right panel) according to certain embodiments;

FIG. 16 shows the change in body weight of the mouse models (n=3) over time from which the pHSN was injected to the proposed site of injection according to FIG. 15; same mouse model was injected with saline as a control group; both groups were monitored for 21 days after injection;

FIG. 17 shows fluorescence images of immunostaining of mouse brain sections from saline-injected (control) and pHSN-injected mouse VTA region by an ionized calcium-binding adapter molecule 1 (Iba1) (left column), glial fibrillary acidic protein (GFAP) (middle column), and caspase-3 (right column);

FIG. 18 shows quantification of immunofluorescent staining as shown in FIG. 17 compared to those without pHSN treatment; no significant differences (n.s.).

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present invention provides modified hollow silica nanoparticles, in particular, a surface-modified hollow silica nanoparticle with PEG (pHSN). Said material is a shelled particle or sphere with a hollow core and surface modification by certain reactive groups and dopants. Said material can disperse well in a medium or suspension for cell culture or in vivo administration, oscillate under ultrasound field, and induce stable cavitation is provided. Said material is capable to facilitate, assist, or synergistically enhance in vitro or in vivo ultrasound stimulation to control certain cellular signalling mechanisms and also chronic stimulation to certain targeted brain regions of a subject. Said material is also capable to stay in the site of administration or targeted tissue/regions of ultrasound stimulation for a reasonably long period of time without causing any damage to corresponding tissues/regions and toxicity effect on the subject.

Similar to some conventional microbubbles or nanobubbles, said material can be used as localized ultrasound neuromodulation actuators to enhance ultrasound effects on modulation of certain cellular activities. Said material is mechanically robust to stay longer in target regions, tissues or sites of administration in vivo and even under insonation. Low or substantially no cytotoxicity also makes said material an ideal biomaterial candidate for long-term precise ultrasound neuronal stimulation and for ultrasound treatment of chronic brain diseases including, but not limited to, Alzheimer's disease, Parkinson's disease, epilepsy, depression, and amyotrophic lateral sclerosis. In order to become localized in the target sites/regions/tissues, said material may be formulated into an injectable medium or suspension form for being administered directly to the corresponding target regions of the subject's brain. In certain embodiments, said material is PEGylated to enhance mechanical strengths, dispersibility in certain solutions or media, and longevity of the present material in vivo. The shell structure of said material is also doped with iron (III) to form Fe (III)-SiO₂ shell particles to facilitate biodegradation in vivo after administration and enhance the HSN structure.

The following examples will assist better understanding and enablement of the present invention but should not be considered limiting the scope of the present invention.

### EXAMPLES

### Example 1—Synthesis of pHSN and Characterization Thereof

Initially, a 0.2% DETA solution was prepared in ethanol solvent and vortexed slightly to mix. Subsequently, 50 mL of ethanol, 4 mL of 0.2% DETA solution, and 2.5 mL of 0.2-0.5 μm polystyrene beads were vortex-mixed for 1 h to produce cationic polystyrene beads. To generate thinner shells, 250 μL of the 20 mg/mL iron (III) ethoxide solution was added into the DETA/polystyrene beads mixture together with the TMOS/TMPS mixture solution, and vortex mixed for another 5 h, to produce iron (III) doped silica shells (s101). Brown core-shell Fe(III)-SiO₂ particles were collected by centrifugation at 3500 rpm for 5 mins, and washed twice with 20 mL of ethanol (s102), followed by drying in air overnight (s103). After drying, the core-shell particles were grounded physically (s104) prior to calcination in air within a muffle furnace, where the temperature starts from room temperature to 550° C. at 1.5° C. per minute (s105). The grounded, calcinated particles in powdery form were filtered by a 40-μm mesh to yield about 10 mg of the hollow silica nanoparticles (HSN) (s106).

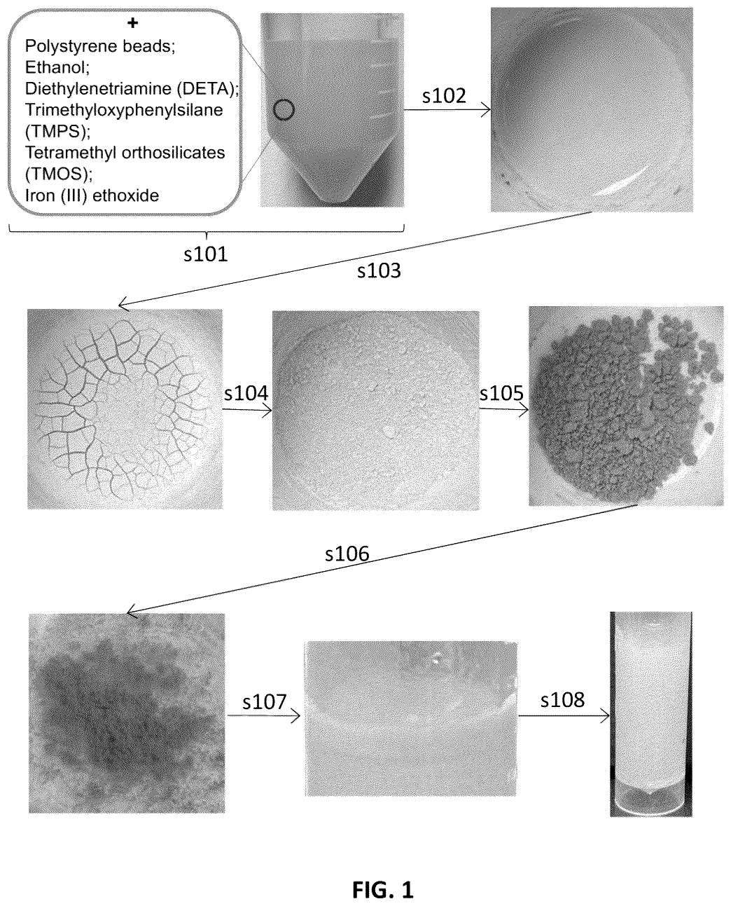

5 mg of the HSN was taken and placed in 2 mL of ethanol solution containing 1.25 μL APTES (3-aminopropyltriethoxysilane), and then stirred at room temperature for 24 h. The mixture was centrifuged at 2000 rpm to collect the precipitate, then washed with 4 times of ethanol. The precipitate was then vacuum dried at room temperature overnight to obtain HSN surface-modified with amine groups (or amino-modified HSN). The amino-modified HSN was mixed with amine-PEG in a molar ratio of 1:1 containing 15 mM 1-(3-dimethylaminopropyl)-3-ethylcarbodiimide hydrochloride (EDC) and 15 mM N-hydroxysuccinimide (NHS) in PBS (pH 7.4) and stirred at room temperature for 24 h. The resulting solution was then centrifugated to remove the excess amount of EDC/NHS and PEG and washed with 4 times of PBS to obtain PEGylated HSN (pHSN) (s107). pHSN was dispersed in PBS as a suspension (s108) before testing or administration in vitro or in vivo. An overall workflow from preparation of HSN to synthesis of pHSN is depicted with corresponding images in FIG. 1. As can be seen from the last image in the workflow of FIG. 1, pHSN powders can disperse very well in PBS, making it highly feasible as an administrable formulation to be biocompatible in most of the animal species.

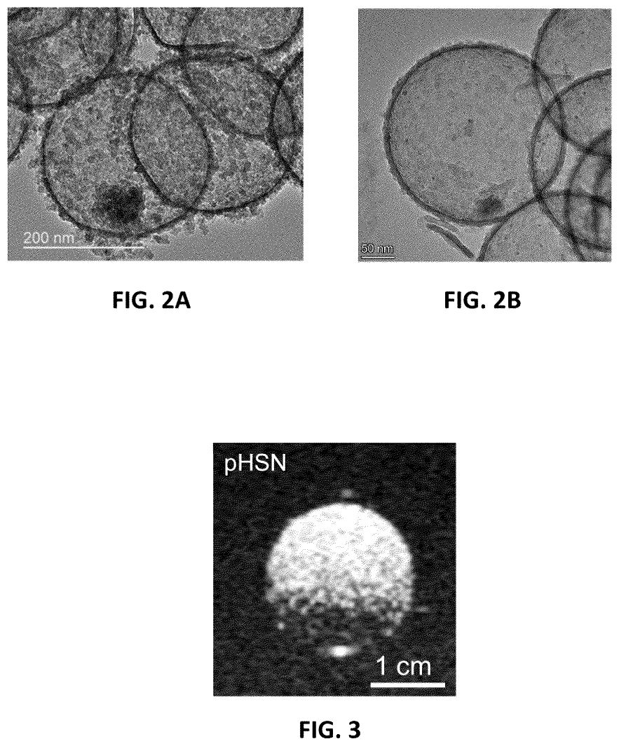

The nanostructure and size of the HSN and pHSN were observed and determined by transmission electron microscopy (TEM) (JEOL 2100 F, JEOL, Tokyo, Japan) operating at 200 kV. pHSN samples were prepared by drop-casting the shells (diluted in ethanol) on lacey carbon-coated copper TEM grids. As seen from FIGS. 2A and 2B, both the HSN and pHSN were substantially spherical with a hollow core, a relatively thin shell structure, and an average diameter from 200 to 300 nm. In particular, the average diameter of pHSN was about 250 nm (FIG. 2A,B).

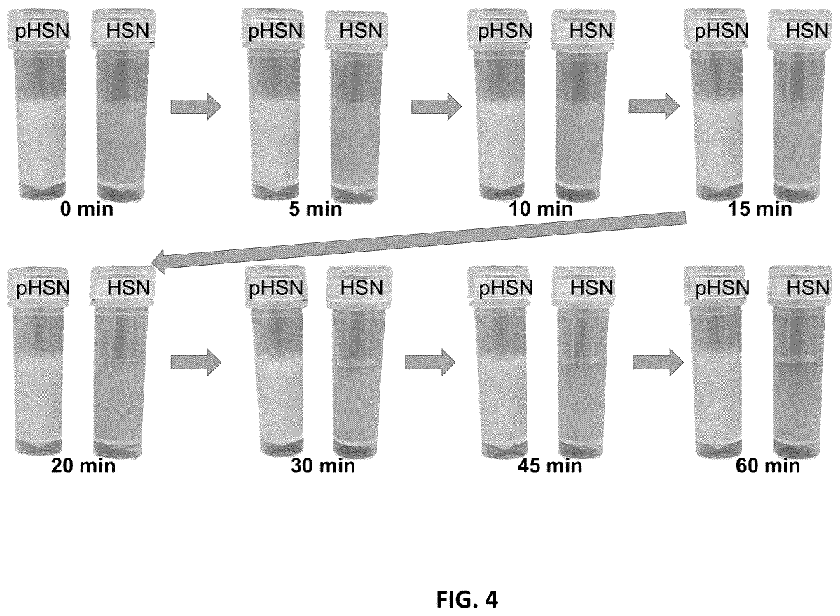

Dispersity of pHSN in PBS solution to form pHSN suspension was also demonstrated in FIG. 4, and PEG surface-modified HSN show a better suspension than the HSN.

### Example 2—Ultrasound Signal Test and Imaging on pHSN

pHSN solution from Example 1 was put into droppers (5 mL) before imaging. Ultrasound B-mode and contrast mode images of the particle solution were acquired using a high-frequency ultrasound system with a transducer of LZ250 D. The center frequency and output energy level were set to 18 MHz and 4%, respectively. FIG. 3 shows a contrast-enhanced ultrasound image of the pHSN.

### Example 3—Passive Cavitation Detection of pHSN In Vitro

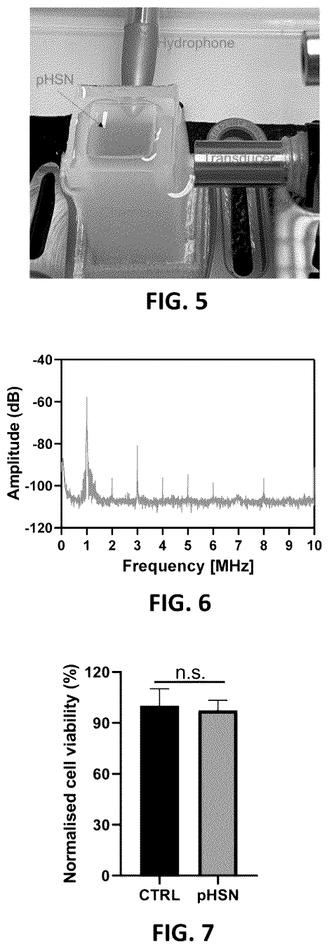

Acoustic spectroscopy on suspensions of pHSN obtained from Example 1 was performed in custom-built chambers, and the 1 MHz flat transducer and hydrophone (HGL-0200, Onda) were perpendicularly aligned and immersed in a tank of deionized, degassed water. An experimental setup of such acoustic spectroscopy on pHSN suspensions is shown in FIG. 5. In accordance with the setup in FIG. 5, a rectangular agarose (3%) chamber with a wall thickness of 5 mm and a cavity having a planar cross-sectional area of 15×15 mm was placed in the middle, with a center point of 17.5 mm away from both the transducer and the hydrophone. 1 MHz sinusoidal trains of burst width 200 μs and burst interval 2 ms were generated by a function generator (AFG251, Tektronix), amplified by a radio frequency amplifier (A075, Electronics & Innovation Ltd.), to drive the emitting transducer, producing acoustic output with 0.28 MPa peak negative pressure (FIG. 5). Signals received by the hydrophone were amplified (AH-2010, Onda) and digitized (CSE1222, GaGe) before analysis. 20 sections of 200 μs digitized signal in 20 separate bursts were processed with fast Fourier transform using MATLAB and the resulting frequency spectra were averaged. The results are shown in FIG. 6.

As seen from FIG. 6, pHSN under ultrasound exposure were able to oscillate in the suspension and backscatter ultrasound signals. The maximum acoustic frequency of the backscattered ultrasound signals by the pHSN appears to fall at 1 MHz based on the acoustic output with a relatively low acoustic intensity (0.3 MPa) by the transducer in this setup. The result accords with the proposed benefits of the present invention from using a low-intensity ultrasound energy with pulsed waves in the presence of the pHSN acting as ultrasound stimulation actuators for amplifying the intensity of the initial ultrasound stimulation signals and maintaining the intensity level while mitigating potential adverse effects such as thermal effects arising from using higher intensity of ultrasound stimulation.

### Example 4—Cytotoxicity Test of pHSN

To evaluate the potential toxicity effect of pHSN on neural system, embryonic rat neurons were seeded in a 96-well plate at a density of 8000 cells per well and cultured overnight at 37° C. in a 5% CO₂ incubator. Before seeding, neurons from rat embryos at embryonic day 18 were obtained according to one of previous studies (Hou et al., “Precise Ultrasound Neuromodulation in a Deep Brain Region Using Nano Gas Vesicles as Actuators”, Adv. Sci., 2021, 8, 2101934, pp 1-12). Initially, cortices were dissected and treated with 0.25% trypsin for 15 min at 37° C., followed by gentle mixing. The digestion was stopped with neurobasal medium (Gibco) with 10% fetal bovine serum and 1% penicillin-streptomycin. The cells were resuspended in medium and gently mechanically triturated with a pipette, and then allowed to stand for 15 min. The resultant supernatant was discarded, and the cells were resuspended in the afore-mentioned medium and plated at PLL-coated glass-bottomed confocal dishes. After 24 h, the medium was changed to neurobasal+2% B27+0.25% L-glutamine+1% penicillin-streptomycin (all from Gibco). Half of the medium was replaced every 2-3 days.

At day-in-vitro (DIV) 10, cells were washed with PBS for 3 times and incubated with solution containing pHSN for 12 h. Cell viability was evaluated by MTT assay. The optical density (OD) was measured at 570 nm and recorded by a microplate reader. The results are shown in FIG. 7.

As seen from FIG. 7, the cell viability in the sample with pHSN was comparable to that without pHSN (CTRL).

### Example 5—Ultrasound Stimulation on Cultured Neurons and Cellular Response Thereof

In this example, a customized system that facilitated ultrasound stimulation and calcium imaging simultaneously on cultured neurons from rat embryos as depicted in Example 4 was used. Briefly, the ultrasound stimulation system was aligned with a calcium imaging system and the calcium responses of the stimulated neurons were monitored. Ultrasound was delivered through a waveguide filled with degassed water that was attached to the ultrasound transducer assembly. 50 μg/mL of pHSN were added to the cell culture medium and gently mixed just before stimulation. Each stimulus was composed of 300 tones burst pulses at a center frequency of 1.0 MHz, 10% duty cycle, and pulse repetition frequency (PRF) of 1 kHz, at low acoustic intensity. These parameters amounted to ultrasound being delivered in very short bursts, minimizing thermal effects.

FIG. 9 shows calcium imaging results before and after ultrasound stimulation on different samples added with or without the pHSN. As seen from FIG. 9, the sample with addition of pHSN after ultrasound stimulation had more calcium influx into the cells (bottom right panel) compared to the same sample before ultrasound stimulation (top right panel). The control samples (without pHSN addition) showed no significant difference in calcium influx before and after ultrasound stimulation (top left and bottom left panels, respectively).

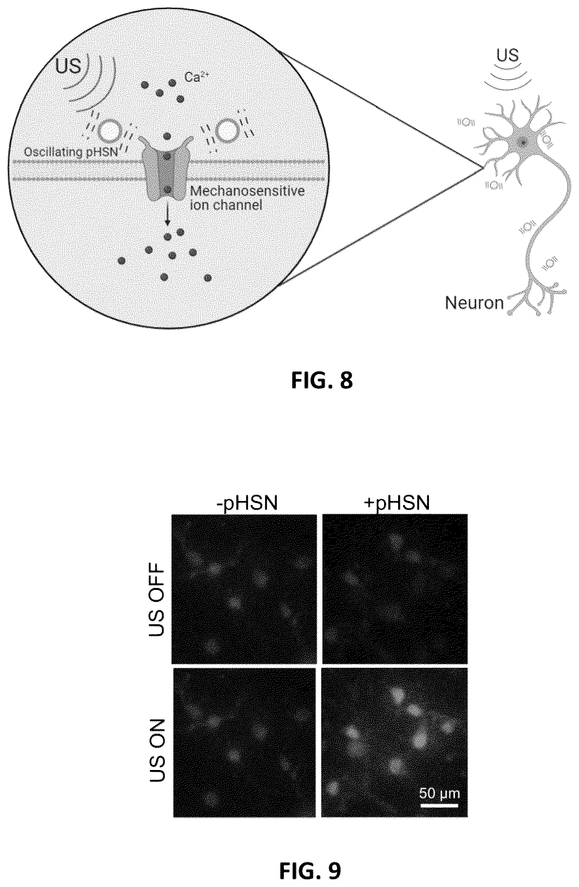

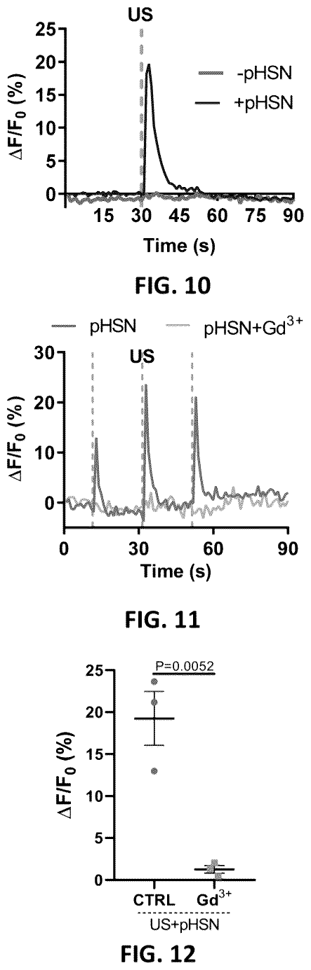

FIG. 10 shows calcium responses of stimulated neuron samples with or without addition of the pHSN. As can be seen, upon ultrasound stimulation, a more significant calcium influx was observed in the sample added with the pHSN (indicated by a sharp change in ΔF/F₀), whereas no change in ΔF/F₀ was observed in the control sample. The results from FIGS. 9-10 correspond to the proposed mechanism of the pHSN in ultrasound neuromodulation depicted in FIG. 8.

Turning to FIGS. 11-12, synchronized neuronal Ca²⁺ responses during three ultrasound stimulations (US, at time intervals indicated by three dotted lines in the plot) on pHSN-treated samples with/without treatment of mechanosensitive ion channels blocker, gadolinium (Gd³⁺), and quantification of the Ca²⁺ responses before and after the Gd³⁺ treatment, are provided. As seen from FIGS. 11-12, the pHSN-treated group without Gd³⁺ treatment had neuronal calcium influx after each of the ultrasound stimulations, whereas the pHSN-Gd³⁺-treated group (HSN+Gd³⁺) had very minimal or substantially zero calcium influx recorded each time after ultrasound stimulations. These results reveal that pHSN provides a mechanical modulation of neuronal responses to ultrasound stimulation.

### Example 6—In Vivo Ultrasound Neuromodulation by pHSN Suspension

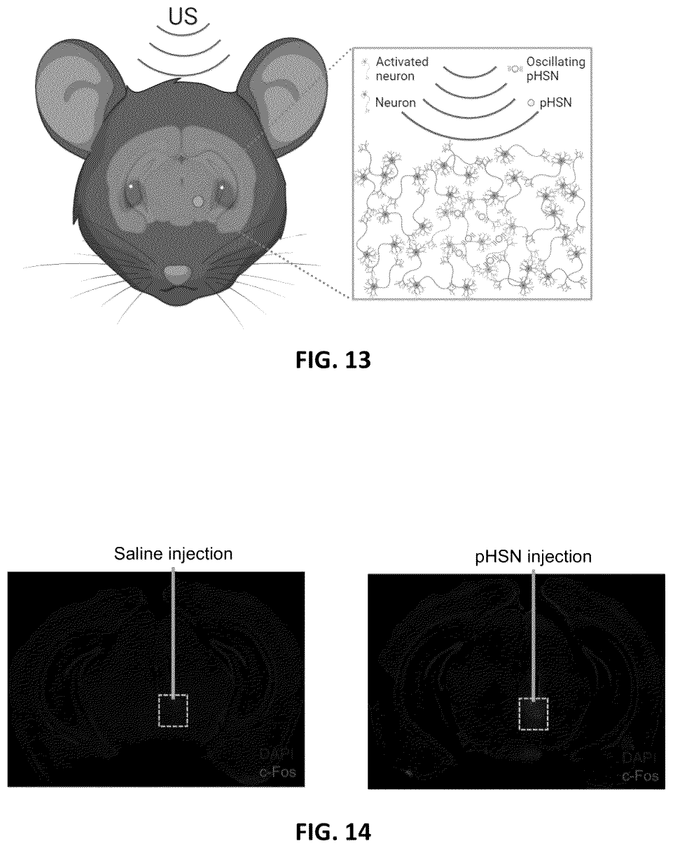

Turning to FIG. 13, a schematic diagram illustrating how the present pHSN modulates neuronal responses to ultrasound stimulation in a brain region of a mouse model in vivo is depicted. 8-week-old C57BL/6 mice were anesthetized with Ketamine and Xylazine (100 and 10 mg/kg, respectively) followed by shaving the skin above a chosen VTA region. By using a stereotaxic apparatus, 1.0 μL of pHSN suspension was injected into the chosen VTA region with coordinates AP: −2.90 mm, ML: −0.50 mm, DV: −4.50 mm. The puncture site was then disinfected and sutured, and the mice were returned to their housing areas. After 1 week of recovery, a 1.0 MHz transducer was coupled to the head with ultrasound gel. Mice were treated with ultrasound for 40 min, after which they were maintained in an anesthetized state for 90 mins.

In the inset on the right panel of FIG. 13, no neurons of the mouse brain were activated by the ultrasound stimulation without pHSN. In contrast, the injected pHSN to the VTA region via the site of injection (indicated by a blue spot on left panel of FIG. 13) were induced to oscillate, sustaining the stimulation around the VTA region such that each instance of ultrasound stimulation did not have to last for a long time, which could be a low-density pulsed ultrasound.

Turning to FIG. 14, low-magnification images of pHSN-injected and saline-injected mice brains are provided, in which the injection site is indicated by the yellow dotted square. On the right panel of FIG. 14, pHSN-injected mouse brain sections showed the pattern of c-Fos expression (red signals), indicating activation of neuronal cells in vivo by the ultrasound stimulation, whereas no c-Fos expression was observed in saline-injected mouse brain sections.

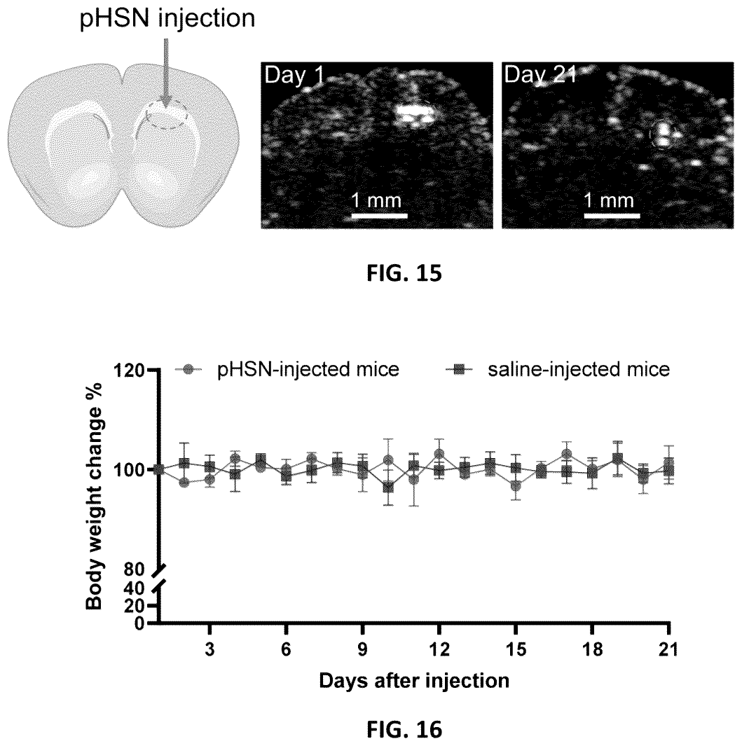

For life-time detection of pHSN in vivo, pHSN suspension was injected into the target site in the brain region of mouse brains. After injection, ultrasound images of mouse brains were recorded on an imaging system (Vevo 2100) at 1, and 21 days. The signal intensities of echo imaging were measured using Vevo 2100 Workstation Software. The results are shown in FIG. 15. From FIG. 15, it can be seen that after 21 days, there were still some signals detected near the site of injection (the blue dotted circles), compared to the other non-injected brain areas.

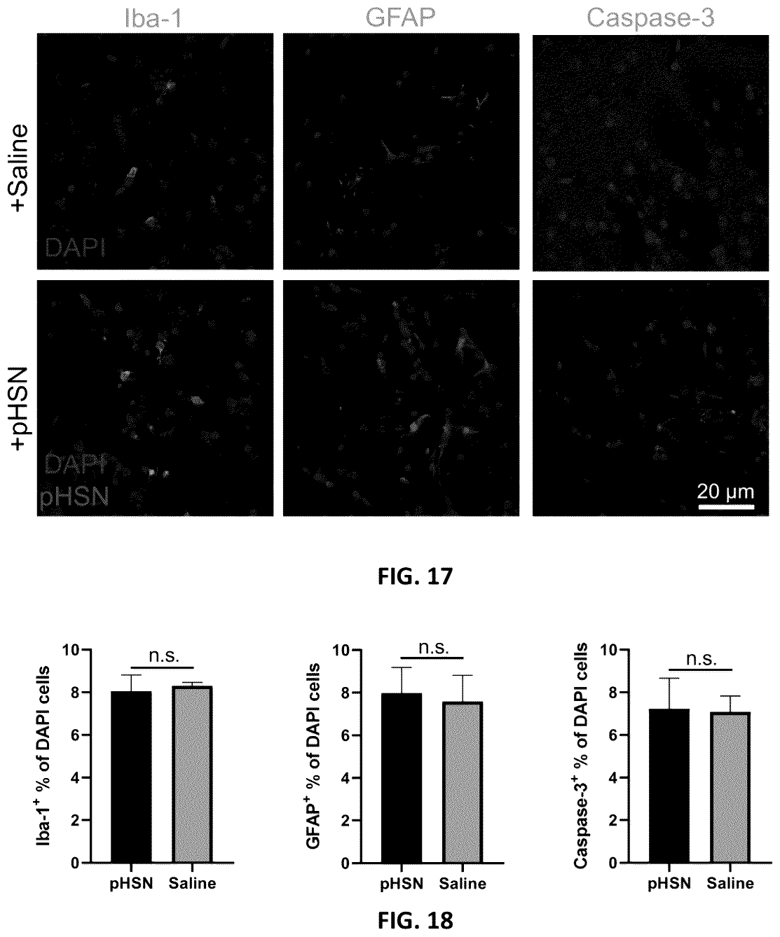

To evaluate the biosafety of pHSN in vivo, the mouse model body weight (FIG. 16) was recorded after the injection of pHSN according to the protocol of the animal study depicted in FIG. 15 (the schematics in the left panel). No obvious body weight change was observed in both the control and pHSN-injected groups over the 21-day duration after injection. The animal brain slices were collected for immunohistochemical staining after several days post-injection. The results are shown in FIG. 17. As seen from FIG. 18, no significant difference in the percentage of microglia (Iba-1 positive), astrocytes (GFAP positive), or apoptotic cells (Caspase-3 positive) was found between the Saline+ and pHSN+ groups, confirming the biosafety of pHSN in vivo.

Comparisons among groups were analyzed via independent-sample one-factor ANOVA test using SPSS 17.0 software. All statistical data shown in the present disclosure including the drawings were obtained using a two-tailed student's t-test and homogeneity of variance tests (p values<0.05 were considered significant).

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. An ultrasound neuromodulation actuator comprising a surface-modified hollow silica nanoparticle (HSN) structure.

2. The ultrasound neuromodulation actuator of claim 1, wherein the surface-modified HSN structure is a PEGylated HSN (pHSN) structure.

3. The ultrasound neuromodulation actuator of claim 1, wherein the surface-modified HSN structure is in particle form with an average diameter from 200 to 300 nm.

4. A method for enhancing ultrasound stimulation in a subject comprising applying the ultrasound neuromodulation actuator of claim 1 to a brain region of the subject and applying an ultrasound stimulation thereafter.

5. The method of claim 4, wherein the ultrasound stimulation has an acoustic intensity of smaller than 0.5 MPa.

6. The method of claim 4, wherein the subject comprises human and non-human animals.

7. A method for treating neurological disorders comprising administering the ultrasound neuromodulation actuator of claim 1 to a subject and applying ultrasound stimulation to one or more regions.

8. The method of claim 7, wherein the surface-modified hollow silica nanoparticle structure is formulated into an injectable formulation and is administered to the subject via intra-VTA administration.

9. The method of claim 7, wherein the one or more regions comprises striatum.

10. The method of claim 7, wherein the neurological disorders comprise Alzheimer's disease, Parkinson's disease, epilepsy, depression, and amyotrophic lateral sclerosis.

11. The method of claim 7, wherein the ultrasound stimulation has an acoustic intensity of smaller than 0.5 MPa.

12. The method of claim 7, wherein the subject comprises primary culture neurons and mice.
